(19) United States
(12) Patent Application Publication
(10) Pub. No.: US 2017/0323696 A1
(43) Pub. Date: Nov. 9, 2017

(54) RADIOPHARMACEUTICAL PRODUCTION SYSTEM, RADIOPHARMACEUTICAL PRODUCTION DEVICE, AND PRODUCTION METHOD FOR RADIOPHARMACEUTICALS

(71) Applicant: HITACHI, LTD., Chiyoda-ku (JP)

(72) Inventors: Yuuko KANI, Tokyo (JP); Takahiro TADOKORO, Tokyo (JP)

(73) Assignee: HITACHI, LTD., Tokyo (JP)

(21) Appl. No.: 15/520,266

(22) PCT Filed: Oct. 14, 2015
(86) PCT No.: PCT/JP2015/079049

§ 371 Date: Apr. 19, 2017

(30) Foreign Application Priority Data
Oct. 20, 2014 (JP) 2014-213609

(51) Int. Cl. G21G 4/08; G21G 1/10; A61K 51/02

(52) U.S. Cl. CPC G21G 4/08; A61K 51/025; G21G 1/10

(57) ABSTRACT

The present invention comprises: an electron beam accelerator (2); a container (4) housing a raw material (3) for radioactive nuclide production, said raw material including molybdenum 100; a heating device (5) that heats the raw material (3) for radioactive nuclide production; an adsorbent (81) that adsorbs technetium compounds including technetium 99m generated by the heated raw material (3) for radioactive nuclide production; an eluent supply device (10) that supplies an eluent (L1) that causes elution of the technetium compound adsorbed to the adsorbent (81); and a drug recovery unit (13) that recovers the eluent (L2).

### TECHNICAL FIELD

The present invention relates to a radiopharmaceutical production system, a radiopharmaceutical production device, and a radiopharmaceutical producing method for producing radiopharmaceuticals using radionuclides.

### BACKGROUND ART

Radiopharmaceuticals are nuclear diagnostic drugs that combine a radionuclide with a drug whose properties make the drug accumulate at the affected region. For example, in SPECT (single photon emission computed tomography), a subject is administered with a radiopharmaceutical combining a radionuclide (for example, technetium 99m) with a drug, and the radiation (gamma rays) emitted by the radionuclide is detected with a camera (gamma camera), and visualized for disease testing. Incidentally, the metastable technetium 99m emits gamma rays by undergoing a nuclear isomer transition to the ground-state technetium 99.

Technetium 99m is a descendant nuclide created by beta decay of the parent radionuclide molybdenum 99, and as such molybdenum 99 is used as a feedstock of radiopharmaceuticals that use technetium 99m. Traditionally, radiopharmaceuticals are produced by a method that uses a molybdenum 99-supporting column, and in which the technetium 99m formed by beta decay of molybdenum 99 is eluted and collected with saline (milking), and a drug is added to the collected technetium 99m to produce a radiopharmaceutical.

Traditionally, molybdenum 99 is produced through nuclear fission of uranium 235, whereby high- or low-enriched uranium 235 in a nuclear reactor is exposed to neutrons, and molybdenum 99 is separated and collected from the nuclear fission product, and purified.

There are only limited numbers of facilities worldwide dedicated to produce molybdenum 99 using such a nuclear reactor, and these are found only in specific locations. Molybdenum 99 has a half-life of about 66 hours, and that of technetium 99m is only about 6 hours. These short half-lives make storage of molybdenum 99 and technetium 99m impossible. Given these circumstances, countries with no molybdenum 99 production facilities must rely on imports by air.

As a response, there are studies of methods for producing radionuclides with use of an accelerator. For example, PTL 1 (WO2011/132265) discloses a method in which molybdenum 100 is bombarded with protons accelerated with an accelerator to produce radionuclides (molybdenum 99, technetium 99m).

There are also methods that collect technetium 99m from molybdenum 99 produced with a nuclear reactor or an accelerator. For example, PTL 2 (JP-A-2011-105567) discloses a method that uses an alumina column supporting molybdenum including molybdenum 99, and saline is passed through the column to separate technetium 99m. PTL 3 (JP-A-2013-35714) discloses a method in which a molybdenum oxide pellet containing molybdenum 99 is dissolved in an alkaline solution, and technetium 99m is extracted and separated using an organic solvent (methyl ethyl ketone). PTL 4 (WO2012/39037) discloses a method in which molybdenum including molybdenum 99 and technetium 99m is dissolved in a solvent, and the solution is passed through a resin-filled column to adsorb technetium to the resin, and separate it from the solution.

### CITATION LIST

### Patent Literature

PTL 1: WO2011/132265
PTL 2: JP-A-2011-105567
PTL 3: JP-A-2013-35714
PTL 4: WO2012/39037

### SUMMARY OF INVENTION

### Technical Problem

The radiopharmaceutical producing methods of the related art are problematic in the following respects.

It is to be noted first that methods that use a nuclear reactor for production of radionuclides for radiopharmaceuticals require large capital investments and high maintenance costs. The method using an accelerator disclosed in PTL 1 can be implemented with smaller devices than methods using a nuclear reactor. However, the method of PTL 1 that involves reaction of accelerated protons and molybdenum requires a middle-sized accelerator to accelerate protons, and there are limitations in miniaturizing the device.

With regard to the technetium 99m (radionuclide) collection method, the method disclosed in PTL 2 is a once-through method so that the molybdenum supported on the alumina column is discarded after use. This is problematic in terms of waste volume. The methods disclosed in PTL 3 and PTL 4 enable collecting molybdenum after technetium separation, and reusing it as an irradiation target. However, the process that produces radiopharmaceuticals by adding a drug after the separation of technetium 99m is inherently a manual procedure requiring an operator, and poses a radiation exposure risk to operators engaged in radiopharmaceutical production.

It is accordingly an object of the present invention to provide a radiopharmaceutical production system, a radiopharmaceutical production device, and a radiopharmaceutical producing method that can be implemented with small devices, and that can reduce the radiation exposure risk to operators engaged in radiopharmaceutical production.

### Solution to Problem

As a solution to the foregoing problems, a radiopharmaceutical production system according to the present invention includes:

a radionuclide production device that produces molybdenum 99 by nuclear reaction through irradiation of a molybdenum 100-containing radionuclide feedstock with radiation generated by using electrons accelerated with an electron beam accelerator; and

a radiopharmaceutical production device that heats the radionuclide feedstock to evaporate a technetium compound containing technetium 99m generated by radioactive decay of molybdenum 99, and adsorbs the evaporated technetium 99m-containing technetium compound to an adsorbent, and in which an eluent is passed through the adsorbent adsorbing the technetium 99m-containing technetium compound, and the technetium 99m-containing technetium compound is eluted into the eluent to produce a radiopharmaceutical.

A radiopharmaceutical production device according to the present invention includes:

an electron beam accelerator;

a container for housing a molybdenum 100-containing radionuclide feedstock to be irradiated with radiation generated by using electrons accelerated with the electron beam accelerator;

a heater for heating the radionuclide feedstock housed in the container;

an adsorbent that adsorbs a technetium compound containing technetium 99m generated upon heating the radionuclide feedstock irradiated with the radiation;

an eluent feeder that supplies an eluent for eluting from the adsorbent the technetium 99m-containing technetium compound adsorbed to the adsorbent; and

a drug collecting section that collects the eluent.

A method for producing a radiopharmaceutical according to the present invention includes:

producing molybdenum 99 by nuclear reaction through irradiation of a molybdenum 100-containing radionuclide feedstock with radiation generated by using electrons accelerated with an electron beam accelerator;

heating the radionuclide feedstock to evaporate a technetium compound containing technetium 99m produced by radioactive decay of molybdenum 99;

adsorbing the evaporated technetium 99m-containing technetium compound to an adsorbent; and

passing an eluent through the adsorbent adsorbing the technetium 99m-containing technetium compound, and eluting the technetium 99m-containing technetium compound into the eluent to produce a radiopharmaceutical.

### Advantageous Effects of Invention

The present invention can provide a radiopharmaceutical production system, a radiopharmaceutical production device, and a radiopharmaceutical producing method that can be implemented with small devices, and that can reduce the radiation exposure risk to operators engaged in radiopharmaceutical production.

### BRIEF DESCRIPTION OF DRAWINGS

FIG. 1 is a schematic view showing a configuration of a radiopharmaceutical production system according to First Embodiment.

FIG. 2 is a schematic view showing a configuration of the inner structure of a radionuclide separation and drug producing unit.

FIG. 3 is a schematic view representing a configuration of a radiopharmaceutical production system according to Second Embodiment.

### DESCRIPTION OF EMBODIMENTS

Modes for carrying out the present invention (hereinafter, referred to as “embodiments”) are described below in detail with reference to the relevant drawings. In the appended drawings, common members are given the same reference numerals, and explanations of such members may not be repeated.

### First Embodiment

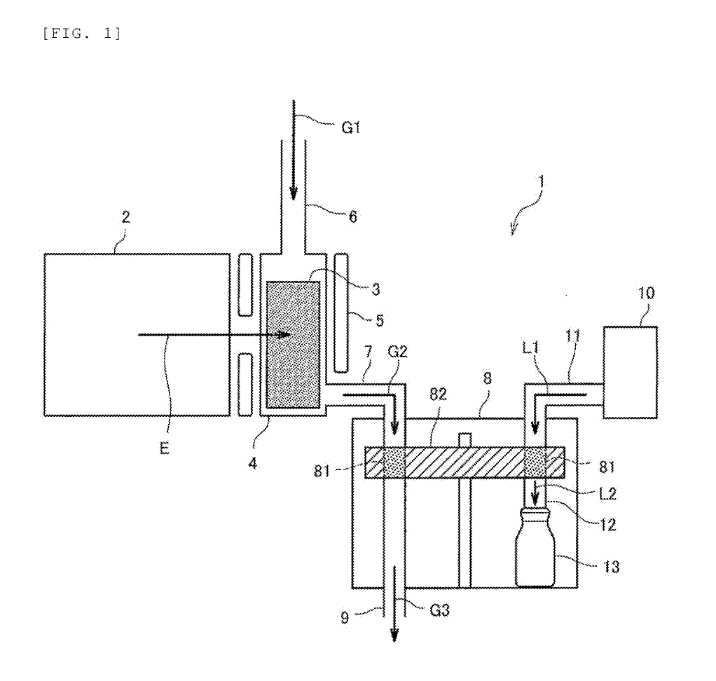

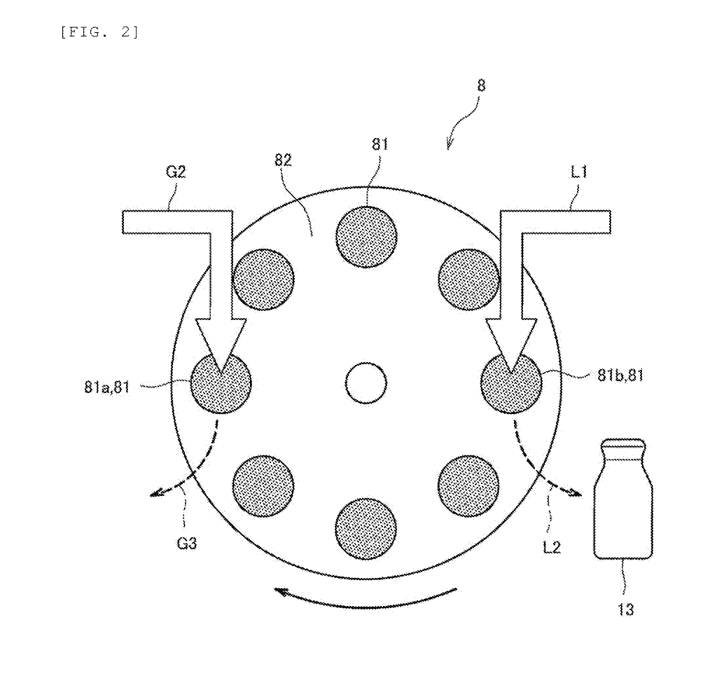

A radiopharmaceutical production system 1 according to First Embodiment is described below with reference to FIGS. 1 and 2. FIG. 1 is a schematic view showing a configuration of the radiopharmaceutical production system 1 according to First Embodiment.

As shown in FIG. 1, the radiopharmaceutical production system (radiopharmaceutical production device) 1 includes an accelerator 2, a heating container 4 for housing a radionuclide feedstock 3, a heater 5, a radionuclide separation and drug producing unit 8, an eluent feeder 10, a radiopharmaceutical collecting section 13, and pipes (6, 7, 9, 11, 12).

The accelerator 2 is an electron beam accelerator, and functions to accelerate electrons. Because the electron has a smaller mass than the proton and heavy particles (e.g., deuteron), the accelerator 2 for accelerating electrons can be made smaller in size than proton accelerators (see PTL 1) for a given acceleration energy.

An electron beam E accelerated by the accelerator 2 falls on the radionuclide feedstock 3 filling the heating container 4. The high-speed electron beam E collides with the radionuclide feedstock 3, and produces bremsstrahlung radiation (electromagnetic rays, or, more specifically, gamma rays) through bremsstrahlung (breaking radiation). Because bremsstrahlung radiation occurs at or near the radionuclide feedstock 3, the radionuclide feedstock 3 becomes irradiated with the bremsstrahlung radiation as it occurs.

In FIG. 1, the accelerator 2 is described as emitting electron beam E to the radionuclide feedstock 3 to expose the radionuclide feedstock 3 to the generated bremsstrahlung radiation. However, the accelerator 2 is not limited to this configuration. A target that generates bremsstrahlung radiation (not illustrated) may be installed at the exit of the accelerator 2, and may be irradiated with an electron beam to generate bremsstrahlung radiation for irradiation of the radionuclide feedstock 3.

The radionuclide feedstock 3 is a molybdenum metal containing molybdenum 100, an isotope of molybdenum. It is also possible to use molybdenum trioxide. The amount of the radionuclide produced by nuclear reaction increases as the molybdenum 100 content in the radionuclide feedstock 3 increases.

The reaction ((γ, n) reaction) between molybdenum 100 and gamma rays (bremsstrahlung radiation) produces molybdenum 99. Molybdenum 99 is a radionuclide with a half-life of about 66 hours, and undergoes radioactive decay (beta decay) to produce technetium 99m (half-life: about 6 hours). The technetium 99m is the radionuclide used herein for the production of radiopharmaceuticals.

The heating container 4 is a container for housing the radionuclide feedstock 3, and is connected to a gas supply pipe 6 and a gas pipe 7. The heater 5 is adapted to heat the heating container 4, and thus the radionuclide feedstock 3 charged inside the heating container 4.

Upon being irradiated with the electron beam E (bremsstrahlung radiation) from the accelerator 2, the radionuclide feedstock 3 inside the heating container 4 becomes a mixture of unreacted molybdenum 100, the (γ, n) reaction product molybdenum 99, and the beta decay product technetium 99m. In the radiopharmaceutical production system 1 according to First Embodiment, technetium 99m is separated from the mixture of molybdenum 100, molybdenum 99, and technetium 99m through evaporative separation that takes advantage of a boiling point difference.

The metal molybdenum has a melting point of 2,623° C., whereas molybdenum trioxide (MoOs) has a melting point of 795° C., and a boiling point of 1,155° C. The metal technetium has a melting point of 2,204° C., whereas technetium oxide (ditechnetium heptoxide; Tc₂O₇) has a melting point of 119.5° C., and a boiling point of 310.6° C.

It is therefore possible to evaporate and separate only the technetium oxide (technetium compound) containing technetium 99m from the mixture (radionuclide feedstock 3) inside the heating container 4 by adjusting the temperature inside the heating container 4 with the heater 5 in a temperature range of not less than 310.6° C.—the boiling point of technetium oxide—and less than 795° C.—the melting point of molybdenum trioxide.

The gas supply pipe 6 is provided to supply a feeding gas G1 into the heating container 4. With the feeding gas G1, the technetium oxide evaporated in the heating container 4 is transported to the radionuclide separation and drug producing unit 8 through the gas pipe 7.

Preferably, the feeding gas G1 is oxygen gas, or a mixed gas of oxygen gas and inert gas. When the radionuclide feedstock 3 is the metal molybdenum, the metal technetium 99m is produced through (γ, n) reaction and beta decay. By supplying the feeding gas G1 containing oxygen, technetium 99m can be separated from the mixture (radionuclide feedstock 3) in the form of a technetium oxide, and collected in the radionuclide separation and drug producing unit 8 (described later). When the radionuclide feedstock 3 is molybdenum trioxide, the collection rate of technetium 99m also can improve by supplying the feeding gas G1 containing oxygen.

The gas pipe 7 is a pipe connecting the heating container 4 to the radionuclide separation and drug producing unit 8, allowing passage of a technetium compound-containing gas G2.

The configuration of the radionuclide separation and drug producing unit 8 is described below with reference to FIG. 2, along with FIG. 1. FIG. 2 is a schematic view showing a configuration of the inner structure of the radionuclide separation and drug producing unit 8.

As shown in FIGS. 1 and 2, the radionuclide separation and drug producing unit 8 includes an adsorbent 81, and an adsorbent transport unit 82. The radionuclide separation and drug producing unit 8 is connected to the heating container 4 via the gas pipe 7, to an offgas processing system (not illustrated) via an offgas pipe 9, to the eluent feeder 10 via the liquid supply pipe 11, and to the radiopharmaceutical collecting section 13 via the liquid pipe 12.

The adsorbent transport unit 82 is a disc-like member that is rotatable about the central axis, and has, for example, a plurality of circular through-holes along the disc circumference and through the top to the bottom surface of the disc, for example, as shown in FIG. 2. The through-holes are charged with the adsorbent 81, which is capable of efficiently adsorbing the technetium compound containing technetium 99m, and allows easy elution of the technetium compound with an eluent (for example, saline) to be described later.

Examples of the adsorbent 81 capable of efficiently adsorbing the technetium compound (technetium oxide) containing technetium 99m include fibrous quartz, alumina, silica gel, organic material fibers such as cotton and nylon, activated carbon, and ion-exchange resins.

In the through-holes of the adsorbent transport unit 82 supporting the adsorbent 81, the through-hole for an adsorbent 81a is connected to the gas pipe 7 on the upper side, and to the offgas pipe 9 on the lower side (see FIG. 1). Specifically, the adsorbent 81a lies on the path leading to the offgas pipe 9 from the gas pipe 7. In the through-holes of the adsorbent transport unit 82 supporting the adsorbent 81, the through-hole for the adsorbent 81b is connected to the liquid supply pipe 11 on the upper side, and to the liquid pipe 12 on the lower side (see FIG. 1). Specifically, the adsorbent 81b lies on the path leading to the liquid pipe 12 from the liquid supply pipe 11. The adsorbent transport unit 82 can be rotated to move the adsorbent 81 from the adsorbent 81a position to the adsorbent 81b position. Specifically, the adsorbent transport unit 82 can move the adsorbent 81a to the adsorbent 81b position. Similarly, the adsorbent 81b can be moved to the adsorbent 81a position.

With this configuration, the technetium compound-containing gas G2 through the gas pipe 7 is supplied to the through-hole charged with the adsorbent 81 (81a), upon which the technetium compound is adsorbed by the adsorbent 81 (81a). Other gases (the oxygen gas accompanying the technetium compound, or a mixed gas of oxygen gas and inert gas, or a gas containing compounds other than the technetium compound generated in the heating container 4) pass through the adsorbent 81 (81a), and moves through the offgas pipe 9 as an offgas G3 before being supplied to the offgas processing system (not illustrated) and processed.

Upon the adsorbent 81 (81a) adsorbing a certain quantity of the technetium compound containing technetium 99m, the adsorbent transport unit 82 is rotated to move the adsorbent 81 (81a) to the adsorbent 81b position. Specifically, the through-hole charged with the adsorbent 81 (81a) is disconnected from the gas pipe 7 and the offgas pipe 9, and connected to the liquid supply pipe 11 and the liquid pipe 12.

As the through-hole charged with the adsorbent 81 (81a) is connected to the liquid supply pipe 11 and the liquid pipe 12, a through-hole charged with a different adsorbent 81 is connected to the gas pipe 7 and the offgas pipe 9. In this way, the process of adsorbing the technetium compound to the adsorbent 81 (81a) can be continuously performed with the process of dissolving the technetium compound from the adsorbent 81 (81b) (the latter process will be described later).

The eluent feeder 10 stores an eluent (for example, saline), and can supply a technetium-compound eluting eluent L1 to the through-hole charged with the adsorbent 81 (81b), via the liquid supply pipe 11.

With this configuration, the eluent L1 through the liquid supply pipe 11 is supplied to the through-hole charged with the adsorbent 81 (81b), after the through-hole charged with the adsorbent 81 (81b) is connected to the liquid supply pipe 11 and the liquid pipe 12. Here, the technetium compound adsorbed by the adsorbent 81 dissolves in the eluent L1, and an eluent L2 dissolving the technetium compound discharges out of the through-hole for the adsorbent 81 (81b) into the radiopharmaceutical collecting section 13 via the liquid pipe 12.

The radiopharmaceutical collecting section 13 contains a drug needed for radiopharmaceutical production (a drug with properties that make the drug accumulate at the affected region). The drug becomes mixed with the eluent L2 containing the technetium compound eluted from the adsorbent 81, and reacts (binds) with the technetium to produce a radiopharmaceutical.

As described above, the radiopharmaceutical production system (radiopharmaceutical production device) 1 according to First Embodiment uses an electron beam accelerator as the accelerator 2, and the series of processes from radionuclide production to radiopharmaceutical production can be performed with smaller devices than in the radiopharmaceutical production system (radiopharmaceutical production device) using a proton accelerator disclosed in PTL 1.

In the radiopharmaceutical production system (radiopharmaceutical production device) 1 according to First Embodiment, the heating temperature for the heating container 4 is set to a temperature that evaporates the technetium compound containing technetium 99m, and accordingly the molybdenum compound containing molybdenum 100 and molybdenum 99 remains in the heating container 4 without evaporating, allowing the radionuclide feedstock 3 to be continuously used. The amount of generated waste is accordingly smaller than in PTL 2.

The radiopharmaceutical production system (radiopharmaceutical production device) 1 according to First Embodiment enables automating the series of processes from radionuclide production to radiopharmaceutical production, and this reduces the radiation exposure risk to operators of drug production as compared to PTL 3 and PTL 4.

The embodiment has been described through the case where the drug needed for radiopharmaceutical production is charged beforehand inside the radiopharmaceutical collecting section 13, and radiopharmaceuticals are produced in the radiopharmaceutical collecting section 13. However, the invention is not limited to this.

For example, the system may be configured so that the drug needed for radiopharmaceutical production is supported on the adsorbent 81 in advance. In such a configuration, the technetium compound and the drug react to produce a radiopharmaceutical as the saline (eluent L1) supplied from the eluent feeder 10 passes through the adsorbent 81 (81b).

The radiopharmaceutical is then collected in the radiopharmaceutical collecting section 13 via the liquid pipe 12.

It is also possible to adopt a configuration in which the drug needed for radiopharmaceutical production is mixed beforehand with the saline supplied from the eluent feeder 10. In such a configuration, the technetium compound reacts with the drug as the technetium compound is eluted from the adsorbent 81 (81b), and produces a radiopharmaceutical. The radiopharmaceutical is then collected in the radiopharmaceutical collecting section 13 via the liquid pipe 12.

The embodiment has been described through the case where the adsorbent transport unit 82 of the radionuclide separation and drug producing unit 8 is rotatable about the central axis, and that the adsorbent 81 is moved from the adsorbent 81a position to the adsorbent 81b position with this structure. However, the invention is not limited to this. Specifically, the transport mechanism is not limited, as long as the adsorbent transport unit 82 at least allows the adsorbent 81 to be moved from the path leading to the offgas pipe 9 from the gas pipe 7 that receives the technetium compound-containing gas G2, to the path leading to the liquid pipe 12 from the liquid supply pipe 11 that receives the eluent L1. For example, the adsorbent 81 may be provided in a cartridge form that is replaceable by a remote operation. It is also possible to adopt a configuration in which the pipe connections are switched so that the adsorbent 81 is moved from the path connecting the gas pipe 7 to the offgas pipe 9, to the path connecting the liquid supply pipe 11 to the liquid pipe 12.

In the radiopharmaceutical production system (radiopharmaceutical production device) 1 according to First Embodiment, a radiation detector (not illustrated) capable of gamma ray detection may be provided near the adsorbent 81a that adsorbs the technetium compound, specifically near the junction connecting the gas pipe 7 to the through-hole supporting the adsorbent 81, or near the junction connecting the offgas pipe 9 to the through-hole supporting the adsorbent 81. With such a configuration, it is possible to check whether the adsorbent 81 (81a) has adsorbed a technetium compound containing a predetermined amount of technetium 99m. The radiation detector (not illustrated) capable of gamma ray detection may be, for example, a NaI detector, or a semiconductor detector.

### Second Embodiment

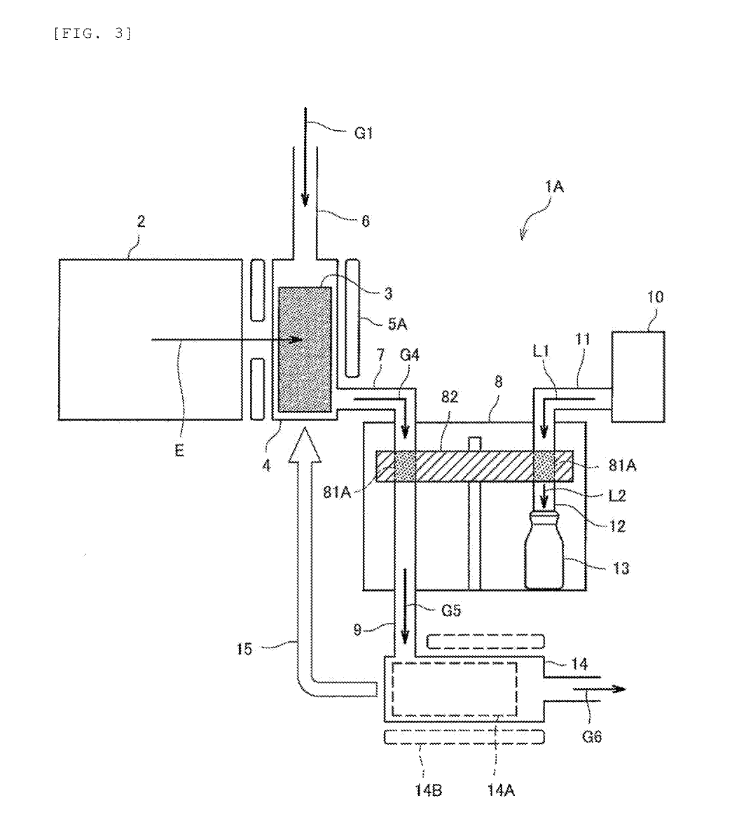

A radiopharmaceutical production system (radiopharmaceutical production device) 1A according to Second Embodiment is described below with reference to FIG. 3. FIG. 3 is a schematic view showing a configuration of the radiopharmaceutical production system 1A according to Second Embodiment.

The radiopharmaceutical production system (radiopharmaceutical production device) 1A according to Second Embodiment (FIG. 3) differs from the radiopharmaceutical production system (radiopharmaceutical production device) 1 according to First Embodiment (FIG. 1) in a heater 5A and an adsorbent 81A. Another difference is the further provision of a feedstock collecting section 14, and a feedstock re-feeding means 15. Other configuration is the same as the radiopharmaceutical production system (radiopharmaceutical production device) 1 according to First Embodiment, and will not be described.

The heater 5A is adapted to heat the heating container 4, and thus the radionuclide feedstock 3 filling the heating container 4. The heater 5A is adjusted so that the temperature inside the heating container 4 becomes a temperature equal to or greater than the sublimation temperature (about 700° C.) of molybdenum trioxide. The temperature inside the heating container 4 is preferably less than the boiling point, 1,155° C., of molybdenum trioxide. Specifically, the temperature is adjusted between 800° C. and 900° C.

With such a configuration, the molybdenum trioxide in the mixture of molybdenum 100, molybdenum 99, and technetium 99m liquefies or sublimes when evaporating the technetium 99m-containing technetium oxide (technetium compound) from the mixture (radionuclide feedstock 3) generated in the heating container 4 by irradiation with the accelerator 2, and the technetium compound can be desirably separated from the mixture. Here, the technetium 99m-containing technetium oxide (technetium compound) generated in the heating container 4 evaporates with the molybdenum trioxide (molybdenum compound) containing molybdenum 100 and molybdenum 99.

Accordingly, a gas G4 containing the technetium compound and the molybdenum compound passes through the gas pipe 7, and flows into a through-hole charged with the adsorbent 81A. Here, an adsorbent capable of selectively adsorbing the technetium compound (technetium oxide) is used as the adsorbent 81A.

Examples of the adsorbent 81 capable of selectively adsorbing the technetium 99m-containing technetium compound (technetium oxide) include activated carbon, and ion-exchange resins.

With such a configuration, the technetium compound- and molybdenum compound-containing gas G4 is supplied through the gas pipe 7 into a through-hole charged with the adsorbent 81A, upon which the technetium compound is adsorbed to the adsorbent 81A. Other gases (the oxygen gas accompanying the technetium compound, a mixed gas of oxygen gas and inert gas, a molybdenum compound gas, or a gas containing compounds other than the technetium compound and the molybdenum compound generated in the heating container 4) pass through the adsorbent 81A, and move through the offgas pipe 9 as a molybdenum compound-containing offgas G5 before being supplied to the feedstock collecting section 14.

In the feedstock collecting section 14, the molybdenum compound (molybdenum trioxide) to be reused as radionuclide feedstock 3 is collected from the molybdenum compound-containing offgas G5, and the offgas G6 is discharged. The offgas G6 is supplied to the offgas processing system (not illustrated), and processed.

Specifically, the feedstock collecting section 14 includes an adsorbent 14A that adsorbs the molybdenum compound (molybdenum trioxide), and collects the molybdenum compound from the molybdenum compound-containing offgas G5. Examples of the adsorbent 14A that adsorbs the molybdenum compound (molybdenum trioxide) include fibrous quartz, alumina, silica gel, organic material fibers such as cotton and nylon, and PZC (poly-zirconium chloride polymer).

The feedstock collecting section 14 also includes a chiller 14B. With the chiller 14B, the feedstock collecting section 14 cools the molybdenum compound-containing offgas G5 to a temperature less than the melting point of molybdenum trioxide, preferably 100° C. or less, and solidifies the gaseous molybdenum trioxide before collecting it.

The feedstock re-feeding means 15 is adapted so that the molybdenum compound (molybdenum trioxide) collected in the feedstock collecting section 14 can be supplied to the heating container 4, either directly or after being optionally processed into a metal. This enables the collected molybdenum compound to be reused as radionuclide feedstock 3. When the adsorbent 14A is used to collect the molybdenum compound in the feedstock collecting section 14, the molybdenum compound adsorbed to the adsorbent 14A may be supplied to the heating container 4 with the adsorbent 14A, provided that the constituting elements of the adsorbent 14A do not have adverse effects on production of molybdenum 99 through gamma irradiation.

As described above, the radiopharmaceutical production system (radiopharmaceutical production device) 1A according to Second Embodiment evaporates the feedstock molybdenum compound with the technetium compound, and promotes evaporation of technetium 99m. This improves the technetium 99m collection rate, in addition to the effects described in First Embodiment. It is accordingly possible to reduce the cost of radionuclide production, which contributes to reducing the cost of radiopharmaceutical production.

Further, because the evaporated molybdenum compound is collected in the feedstock collecting section 14, and reused as radionuclide feedstock 3 using the feedstock re-feeding means 15, it is possible to reduce waste.

### REFERENCE SIGNS LIST

1, 1A: Radiopharmaceutical production system (radiopharmaceutical production device)
2: Accelerator (electron beam accelerator)
3: Radionuclide feedstock
4: Heating container
5, 5A: Heater
6: Gas supply pipe
7: Gas pipe
8: Radionuclide separation and drug producing unit
81, 81A: Adsorbent
82: Adsorbent transport unit
9: Offgas pipe
10: Eluent feeder
11: Liquid supply pipe
12: Liquid pipe
13: Radiopharmaceutical collecting section
14: Feedstock collecting section (feedstock collector)
14A: Adsorbent
14B: Chiller
15: Feedstock re-feeding means (feedstock re-feeder)
E: Electron beam
G1: Feeding gas
G2: Technetium compound-containing gas
G3: Offgas
G4: Technetium compound- and molybdenum compound-containing gas
G5: Molybdenum compound-containing offgas
G6: Offgas
L1: Eluent
L2: Technetium compound-containing eluent

1. A radiopharmaceutical production system comprising:
a radionuclide production device that produces molybdenum 99 by nuclear reaction through irradiation of a molybdenum 100-containing radionuclide feedstock with radiation generated by using electrons accelerated with an electron beam accelerator; and
a radiopharmaceutical production device that heats the radionuclide feedstock to evaporate a technetium compound containing technetium 99m generated by radioactive decay of molybdenum 99, and adsorbs the evaporated technetium 99m-containing technetium compound to an adsorbent, and in which an eluent is passed through the adsorbent adsorbing the technetium 99m-containing technetium compound, and the technetium 99m-containing technetium compound is eluted into the eluent to produce a radiopharmaceutical.

2. The radiopharmaceutical production system according to claim 1, wherein the molybdenum 100-containing radionuclide feedstock is a molybdenum metal or molybdenum trioxide.

3. The radiopharmaceutical production system according to claim 1, wherein the technetium 99m-containing technetium compound is evaporated under a stream of gas.

4. The radiopharmaceutical production system according to claim 3, wherein the gas is oxygen gas, or a mixed gas of oxygen gas and inert gas.

5. The radiopharmaceutical production system according to claim 1, wherein a saline is passed as the eluent through the adsorbent adsorbing the technetium 99m-containing technetium compound to produce a saline solution of technetium 99m, and the solution is added to a drug for radiopharmaceutical production.

6. The radiopharmaceutical production system according to claim 1, wherein the adsorbent supports a drug for radiopharmaceutical production, and the eluent is passed through the adsorbent adsorbing the technetium 99m-containing technetium compound to synthesize the radiopharmaceutical.

7. The radiopharmaceutical production system according to claim 1, wherein the eluent is a saline containing a drug for radiopharmaceutical production, and the eluent is passed through the adsorbent adsorbing the technetium 99m-containing technetium compound to synthesize the radiopharmaceutical.

8. The radiopharmaceutical production system according to claim 1, wherein the radionuclide feedstock is heated at a temperature that does not evaporate the molybdenum compound but selectively evaporates the technetium compound.

9. The radiopharmaceutical production system according to claim 8, wherein the adsorbent contains any one of fibrous quartz, alumina, silica gel, an organic material fiber, activated carbon, and an ion-exchange resin.

10. The radiopharmaceutical production system according to claim 1, wherein the radionuclide feedstock is heated at a temperature that evaporates the molybdenum compound and the technetium compound.

11. The radiopharmaceutical production system according to claim 10, wherein adsorbent selectively adsorbs the technetium compound from a mixture of the molybdenum compound and the technetium compound.

12. The radiopharmaceutical production system according to claim 8, wherein the molybdenum compound and the technetium compound are oxides.

13. The radiopharmaceutical production system according to claim 10, further comprising:
a feedstock collector that collects the molybdenum compound from an offgas that passed through the adsorbent; and
a feedstock re-feeder that enables the collected molybdenum compound to be reused as the radionuclide feedstock.

14. A radiopharmaceutical production device comprising:
an electron beam accelerator;
a container for housing a molybdenum 100-containing radionuclide feedstock to be irradiated with radiation generated by using electrons accelerated with the electron beam accelerator;
a heater for heating the radionuclide feedstock housed in the container;
an adsorbent that adsorbs a technetium compound, the technetium compound contains technetium 99m, the technetium compound is generated upon heating the radionuclide feedstock irradiated with the radiation;
an eluent feeder that supplies an eluent for eluting from the adsorbent the technetium 99m-containing technetium compound adsorbed to the adsorbent; and
a drug collecting section that collects the eluent.

15. A method for producing a radiopharmaceutical,
the method comprising:
producing molybdenum 99 by nuclear reaction through irradiation of a molybdenum 100-containing radionuclide feedstock with radiation generated by using electrons accelerated with an electron beam accelerator;
heating the radionuclide feedstock to evaporate a technetium compound containing technetium 99m produced by radioactive decay of molybdenum 99;
adsorbing the evaporated technetium 99m-containing technetium compound to an adsorbent; and
passing an eluent through the adsorbent adsorbing the technetium 99m-containing technetium compound, and eluting the technetium 99m-containing technetium compound into the eluent to produce a radiopharmaceutical.
